(19) United States
(12) Patent Application Publication
(10) Pub. No.: US 2023/0118755 A1
(43) Pub. Date: Apr. 20, 2023

(54) GASTRIC OUTFLOW REGULATION DEVICES AND METHODS FOR WEIGHT LOSS

(71) Applicant: Nader Sayed Fahmy, Ancaster (CA)

(72) Inventor: Nader Sayed Fahmy, Ancaster (CA)

(21) Appl. No.: 17/965,949

(22) Filed: Oct. 14, 2022

Related U.S. Application Data
(60) Provisional application No. 63/256,125, filed on Oct. 15, 2021.

(51) Int. Cl. A61F 5/00

(52) U.S. Cl. CPC A61F 5/0079; A61F 5/0046; A61F 5/003

(57) ABSTRACT

A gastric outflow device is described. The gastric outflow device includes a ring having a ring aperture; and a topper having a topper aperture, the topper attachable to the ring, the topper aperture alignable with the ring aperture to form the gastric outflow device aperture. The gastric outflow device aperture can be adjusted wirelessly, percutaneously, by replacing the topper, and/or by including an expandable compartment surrounding the aperture, the expandable compartment increasing and decreasing the gastric outflow device aperture on being filled with filling material and having the filling material extracted, respectively.

### FIELD

The present specification relates generally to control of gastric outflow, and, in particular, to devices for restricting gastric outflow such as to help with weight loss.

### BACKGROUND

Human overeating and excessive weight gain are common maladies where access to food is plentiful and lifestyles are particularly sedentary. Overeating and overweight humans produce disproportionate amounts of food waste and are at higher risk of multiple health concerns. This can place significant strain on the environment, agriculture, waste, and health care systems. There are health and systemic benefits to reducing food consumption and maintaining healthy body weights. As a result, a number of weight control and weight loss mechanisms have become known.

For example, restricting caloric intake, otherwise known as “dieting”, is a known method for weight control and weight loss; however, it requires a great deal of motivation in order to succeed and hence may typically fail. Other methods, such as surgical interventions, build on the theory of caloric restriction and endeavor to physically alter the human anatomy in order to restrict the intake of food. These known surgical methods include, for example, bariatric surgery, gastric band surgery, and gastric balloons. These methods are often expensive, irreversible, highly invasive and often unsuccessful in maintaining one's weight. Further, not everyone qualifies for these invasive measures, as one's body mass index (BMI) must be particularly high and, even if they do qualify, they often choose not to undergo the procedure because of associated risks, costs, and low efficacy.

### SUMMARY

In accordance with an aspect, a gastric outflow device includes a ring having a ring aperture; and a topper having a topper aperture, the topper attachable to the ring, the topper aperture alignable with the ring aperture to form the gastric outflow device aperture.

In some embodiments, the topper includes screws spaced near an outer perimeter of the topper, the ring comprising holes alignable for receiving the screws.

In some embodiments, the topper further includes protrusions spaced along a surface of the topper, the ring further comprising grooves alignable for receiving the protrusions.

In some embodiments, the screws are evenly spaced along the outer perimeter of the topper and the protrusions evenly spaced between the screws.

In some embodiments, the gastric outflow device aperture extends to an outer perimeter of the gastric outflow device.

In some embodiments, the gastric outflow device aperture defines a portion of an outer perimeter of the gastric outflow device.

In some embodiments, the topper aperture is adjustable by wireless signal.

In some embodiments, the topper aperture is adjustable by percutaneous control.

In some embodiments, the topper aperture is adjustable by an ultrasonic motor.

In some embodiments, the ring further includes staples attachable to a pyloric sphincter and the topper further comprising screws securable to the ring.

In some embodiments, further includes an expandable compartment expandable by being filled with filling material and contractable by extraction of the filling material, the expandable compartment positioned in the gastric outflow device aperture and comprising an expandable compartment aperture.

In some embodiments, the gastric outflow device further includes a conduit in fluid communication with the expandable compartment, the conduit including a valve through which the filling material is insertable and extractable.

In some embodiments, the gastric outflow device further includes at least one support attachable to an inner perimeter of the expandable compartment and to a separate portion of the expandable compartment, the inner perimeter defining the expandable compartment aperture, the at least one support directing expansion or contraction of the expandable compartment radially.

In some embodiments, the gastric outflow device further includes a cross-linking structure attachable to opposing surfaces of the expandable compartment, the cross-linking structure restricting movement of the expandable compartment axially.

In accordance with an aspect, a method for restricting gastric outflow includes directing gastric outflow through an aperture sized and dimensioned according to a pre-determined level of gastric outflow desired.

In some embodiments, the method further includes adjusting the size of the aperture based on an adjusted pre-determined level of gastric outflow desired.

In some embodiments, the adjusting further includes an elastic adjustment by filling or extracting filling material surrounding the aperture.

In some embodiments, the method further includes replacing the aperture with a replacement aperture sized and dimensioned according to an adjusted pre-determined level of gastric outflow desired.

In some embodiments, the method further includes adjusting the size of the aperture using wireless control.

In some embodiments, the method further includes adjusting the size of the aperture using percutaneous control.

In accordance with an aspect, a gastric outflow device includes a flexible conduit wrappable around a bodily conduit, the flexible conduit configured to expand to tighten around the bodily conduit following receiving a filling material, the flexible conduit configured to contract to loosen around the bodily conduit following extraction of the filling material.

In some embodiments, there is provided a gastric outflow device, comprising a ring, wherein the ring is defined by a concentric outer and inner perimeter and wherein the ring is substantially circular and configured to secure within a human pyloric sphincter; a topper, wherein the topper is defined by a concentric outer and inner perimeter and wherein the ring is substantially circular and configured to secure to the ring; and an aperture, wherein the aperture is defined by the inner perimeter of the topper and wherein the aperture is substantially circular and configured to receive gastric outflow from a stomach.

Other aspects and features according to the present application will become apparent to those ordinarily skilled in the art upon review of the following description of embodiments of the invention in conjunction with the accompanying figures.

### BRIEF DESCRIPTION OF THE DRAWINGS

The principles of the invention may better be understood with reference to the accompanying figures provided by way of illustration of an exemplary embodiment, or embodiments, incorporating principles and aspects of the present invention, and in which:

FIG. 1 is an anterior view of a ring, according to some embodiments, attached to a pyloric sphincter;

FIG. 2 is a front view of a topper, according to some embodiments;

FIG. 3 is a back view of the topper of FIG. 2;

FIG. 4 is a side view of the topper of FIG. 2;

FIG. 5 is a front view of a topper, according to some embodiments;

FIG. 6 is an anterior, cross-sectional view of a stomach, with an inserted gastric outflow device, according to some embodiments;

FIG. 7 is an internal, lateral, cross-sectional view of a gastric outflow device, according to some embodiments;

FIG. 8 is an anterior, cross-sectional view of a pyloric sphincter, with an inserted gastric outflow device, according to some embodiment;

FIG. 9 is a flow chart depicting the various types of gastric outflow devices;

FIG. 10 is a top view of a bubble included in a gastric outflow device, according to some embodiments;

FIG. 11 is a schematic view of a gastric outflow device, according to some embodiments;

FIG. 12 is a schematic view of a gastric outflow device, according to some embodiments; and

FIG. 13 is a cross-sectional view of two parts of a lock of a gastric outflow device, according to some embodiments.

### DETAILED DESCRIPTION

The description that follows, and the embodiments described therein, are provided by way of illustration of an example, or examples, of particular embodiments of the principles of the present invention. These examples are provided for the purposes of explanation, and not of limitation, of those principles and of the invention. In the description, like parts are marked throughout the specification and the drawings with the same respective reference numerals. The drawings are not necessarily to scale and in some instances, proportions may have been exaggerated in order more clearly to depict certain features of the invention.

Gastric outflow regulation can be targeted pre and post (before and after) the pylorus. Devices can be used separately or conjointly to facilitate weight loss and maintenance.

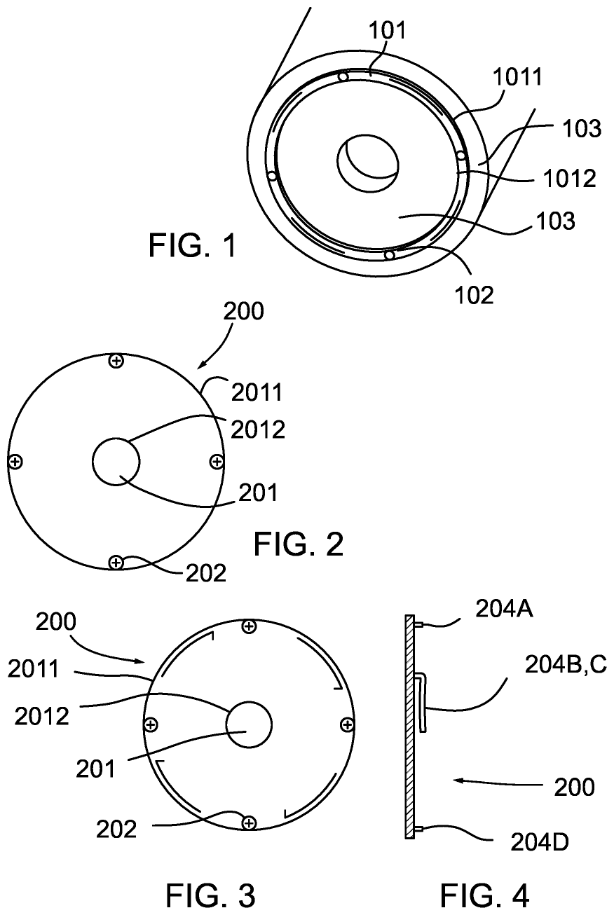

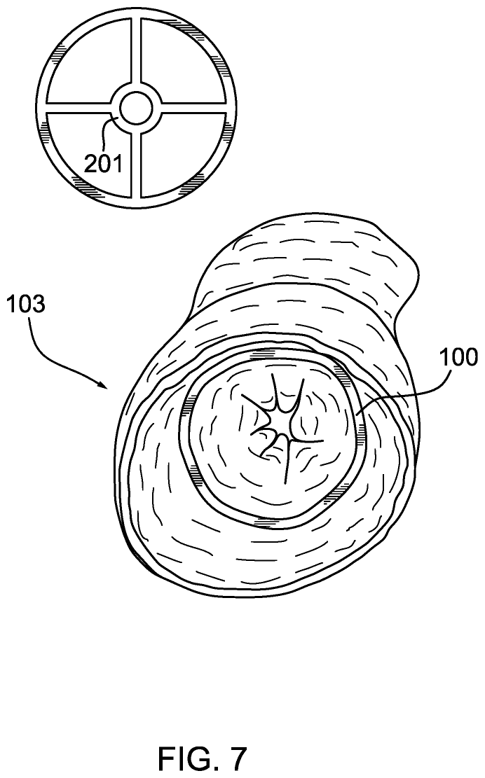

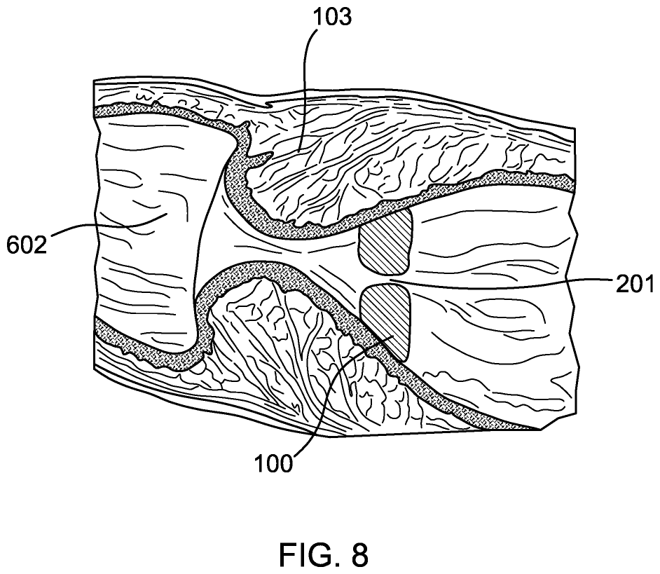

According to embodiments shown in FIGS. 6 to 8, a gastric outflow device 100 is configured to control gastric outflow (i.e., ingested food) from a human stomach 601 to duodenum 602, via an opening in pyloric sphincter 103. By attaching gastric outflow device 100 to a human pyloric sphincter 103, between the stomach 601 and duodenum 602, gastric outflow device 100 reduces the diameter of the opening through which gastric outflow may exit the stomach 601, via pyloric sphincter 103. The reduction in the opening of the pyloric sphincter 103, caused by gastric outflow device 100, leads to an increase in resistance and, as a result, increased pressure in the stomach 601. This increase in pressure in the stomach 601 may result in feelings of fullness and satiation and, therefore, a decrease in one's urge to eat. Long-term use of gastric outflow device 100 may decrease the amount of food regularly ingested and, therefore, result in more permanent reductions in the size of one's stomach 601. Weight loss, as a result of lower food intake, may also occur. Further, over time, as one's caloric intake decreases and/or weight loss goals are obtained, gastric outflow device 100 can be removed or adjusted. For example, an adjustment to gastric outflow device 100 that results in a decrease in diameter through which gastric outflow exits the stomach 601 through gastric outflow device 100, will decrease the amount of gastric outflow. This results in greater pressure exerted on the stomach 601 and, therefore, greater feelings of satiety. Conversely, an increase in a diameter through which gastric outflow exits the stomach 601 through gastric outflow device 100, increases the amount of gastric outflow. This results in less pressure exerted on the stomach 601 and, therefore, weaker feelings of satiety. Ideally, gastric outflow device 100 should be attached such that it initially allows for moderate flow of gastric outflow from the stomach 601 through to one's duodenum. Once attached (e.g., inserted), gastric outflow device 100 may be used to further decrease gastric outflow, as tolerated by the individual, to increase feelings of satiety. In particular, topper 200 (e.g., as shown in FIG. 2) can be removed and replaced with a topper 200 having a different sized aperture 201 and secured to ring 101 (e.g., as shown in FIG. 1). If, after removal of gastric outflow device 100, body weight is regained, gastric outflow device 100 may be reattached. Gastric outflow device 100 may be attached to pyloric sphincter 103 (e.g., as shown in FIG. 1). Gastric outflow device 100 can be installed at the stomach 601 side or at the duodenum side. Gastric outflow device 100 can be oriented in reverse, with a first side facing the stomach 601 side or, alternatively, with the first side facing the duodenum side.

According to an embodiment shown in FIG. 1, gastric outflow device 100 includes a ring 101. Ring 101 may be formed from a single piece of material shaped into an “O”. Ring 101 is defined by an outer perimeter 1011 and inner perimeter 1012. These can be concentric perimeters. Further, gastric outflow device 100 can be configured for attachment to (e.g., such as insertion on) a human pyloric sphincter 103. Ring 101 can be substantially circular, such that outer perimeter 1011 is capable of being secured to pyloric sphincter 103. In some embodiments, ring 101 can be a different shape. In some embodiments, in order to suitably restrict gastric outflow, ring 101 is configured to form an appropriate seal about the circumference of pyloric sphincter 103 and the shape of ring 101 can be configured to facilitate same, such as being configured to be substantially circular in shape. Ring 101 may be secured to pyloric sphincter 103 by staples or another suitable method. Gastric outflow device 100 also includes topper 200 (e.g., as shown in FIG. 2), which may be formed from a single piece of material shaped to couple with ring 101. Topper 200 comprises an outer perimeter 2011 and inner perimeter 2012, and these perimeters can be concentric. In some embodiments, the respective shapes of ring 101 and topper 200 are configured to facilitate attachment to each other. For example, outer perimeter 2011 can be securable to ring 101. Topper 200 may be secured to ring 101 by micro screws 202 and, in some embodiments, by four micro screws 202 evenly disposed near outer perimeter 2011. Ring 101 includes aligned holes 102 for this purpose. Pre-drilled holes 102 and micro screws 202 are alignable, such that micro screws 202 can pass through pre-drilled holes 102 and secure topper 200 to ring 101. This can facilitate a suitable seal between topper 200 and ring 101.

In order to determine the appropriate size of gastric outflow device 100 for each individual, an abdominal ultrasound machine can be utilized in order to measure the circumference of pyloric sphincter 103 and, in particular, the pyloric canal. Gastric outflow device 100 can include receptacles at the outer edge of the device configured for attachment to a sphincter near the stomach 601 and duodenum.

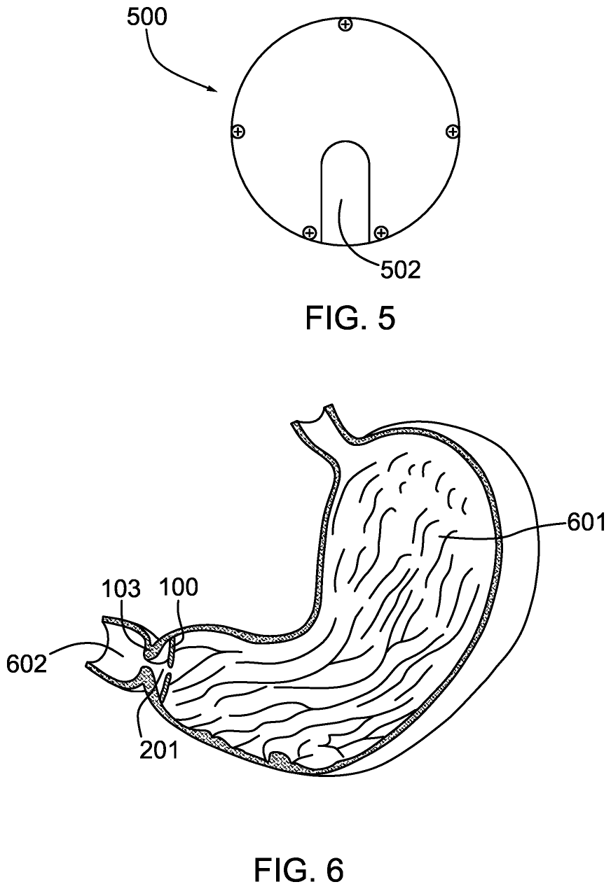

In some embodiments, gastric outflow device 100 does not include a topper 200 having an opening 201 that is adjustable (e.g., by electromagnetic signal or wireless signal), but instead defines an opening of fixed size. In some embodiments, the opening or, in an embodiment having an adjustable opening 201, such opening or aperture 201 is of one of a variety of shapes or configurations. An example of an alternative such opening is shown in FIG. 5. In an embodiment, topper 200 and/or ring 100 may be in another suitable shape other than an “O” or substantially circular shape.

According to embodiments shown in FIGS. 2 to 4, topper 200 also includes an aperture 201, defined by inner perimeter 2012. Aperture 201 is also substantially circular and configured to receive gastric outflow from the stomach 601. In an embodiment, aperture 201 is also adjustable, which enables gastric outflow device 100 to exert various degrees of resistance on gastric outflow exiting the stomach 601 via pyloric sphincter 103. In an embodiment, topper 200 is replaced with a topper 200 having a different sized aperture 201 (e.g., smaller diameter) and this can allow for adjustment of caloric intake and passage of food. For example, a decrease in diameter of aperture 201, decreases the amount of gastric outflow, increases pressure in the stomach 601 and, as a result, can increase feelings of fullness and satiety. On the other hand, an increase in diameter of aperture 201, increases the amount of gastric outflow, decreases pressure in the stomach 601 and, as a result, can lessen feelings of fullness and satiety. Considerations such as target weight and length of time required to achieve target weight are also contemplated when determining the appropriate aperture 201 diameter. Once aperture 201 diameter is determined, a static topper 200 with a static aperture 201 diameter may be secured to ring 101, which has been attachment to (e.g., such as insertion on) pyloric sphincter 103. As various degrees of gastric outflow is desired, topper 200 may be removed and reattached with a different topper 200 having a different aperture 201 of a different diameter. In some embodiments, aperture 201 may be adjusted by percutaneous or wireless control. As non-mutually exclusive examples, aperture 201 may be adjusted by ultrasonic motor, electromagnetic control, and/or external control. Aperture 201 may be adjusted using programming and/or percutaneous control.

In some embodiments, topper 200 includes microscrews 202 on a face of topper 200. As shown in FIG. 4, topper 200 includes protrusions 204A, 204B, 204C, and 204D that extend from the opposite face of topper 200. The protrusions 204A, 204B, 204C, and 204D secure to complementary grooves in ring 101 and can be hook-like, for example. On insertion using a gastroscope, topper 200 is rotated near ring 101 to allow each protrusion 204A, 204B, 204C, and 204D to hook into and secure to ring 101 at complementary grooves. The grooves in 101 can be located at approximately equal distances between holes for receiving microscrews from topper 200, for example, as shown in FIG. 1. This can allow topper 200 to be quickly, accurately, and easily secured with a ring 101 installed at the pyloric valve. Topper 200 can be secured to ring 101 at four hole-microscrew components and four groove-protrusion components. In some embodiments, other securement components and methods can be used to secure topper 200 to ring 101.

Gastric outflow device 100 and, in particular, ring 101 and topper 200, may be constructed from materials which provide sufficient mechanical strength and durability to restrict human pyloric sphincter 602. According to an embodiment, gastric outflow device 100 may be constructed of a polymer that is acid resistant and has elastic properties or a combination of suitable materials. This can include silicone. Manufacturing costs and desired lifespan may be considerations in informing the choice of materials and construction technique and design choices of gastric outflow device 100.

Topper 200 can be formed from a single piece of material shaped into a substantially circular disk.

According to a further embodiment, as shown in FIG. 5, topper 500 contains an aperture 502 may be located along a perimeter of topper 500. In some embodiments, aperture 502 extends to an outer perimeter of gastric outflow device 500. In some embodiments, aperture 502 defines a portion of the outer perimeter of gastric outflow device 500. For example, aperture 502 can define a break in an otherwise substantially circular disk forming topper 500. Aperture 502 can be of a variety of shapes. For example, in the embodiment shown, aperture 502 is substantially oval in shape. A ring complementary to topper 500 functions and is attached in the same ways as in gastric outflow device 100. In some embodiments, aperture 502 can be another suitable shape. The shape of aperture 502 may allow for certain medications to pass through the stomach into duodenum more quickly, as aperture 502 can slow the gastric flow but does not prevent it. In some embodiments, aperture 502 reduces gastric flow but does not delay it. In contrast, aperture 201 requires a particular volume of gastric flow, such that it can fill and pass through the height of aperture 201 on topper 200. In some embodiments, aperture 201 reduces gastric flow and delays it. In an embodiment, a different configuration, number, and alignment of securing features (e.g., staples) can be included in topper and/or with complementary securing features (e.g., holes) in a complementary ring, such as shown in the example topper 500 showing five microscrews.

Gastric flow device 100 may be attached to a human pylorus sphincter by using a gastroscope to place and secure ring 101 to the muscle of a pylorus sphincter. Ring 101 can be secured to the muscle with staples or another suitable method. Once ring 101 is secured, topper, such as topper 200 or 500, is inserted via a gastroscope and micro screws 202 are aligned with pre-drilled holes 102. Once appropriately aligned, micro screws 202 may be fastened to ring 101.

In an embodiment, sutures can be used to permanently reduce the size of a pyloric sphincter to restrict gastric outflow. This can be performed in conjunction with use of gastric outflow device 100.

In an embodiment, gastric flow device 100 can allow for weight loss via caloric intake reduction through gastric outflow regulation, as well as facilitate human carbon footprint reduction and waste reduction and mitigating world hunger crises by reducing food intake to a reasonable healthy level. Collectively reducing food consumption worldwide can have a significant positive impact on the environment.

In the spirit of controlling gastric outflow, techniques to control pyloric sphincter tone, strength, and action via 1) injecting bulking or hypertrophic agents into the sphincter body; 2) controlling blood supply of sphincter muscle to increase or decrease its bulk and/or size; and/or 3) injecting Botox into the sphincter muscular body can be used with gastric outflow device 100.

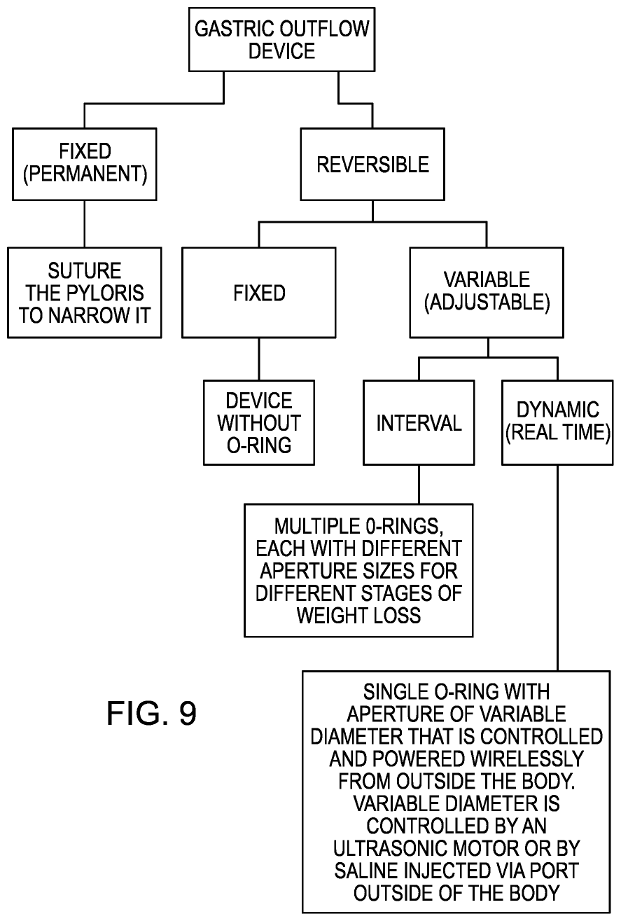

FIG. 9 depicts the characteristics of various types of gastric outflow devices use to restrict gastric outflow. These gastric outflow devices may be pre-pylorus gastric outflow devices. Gastric outflow may be restricted by permanent methods or reversible devices. For example, one permanent method is to narrow the pyloric sphincter 103 by suturing. In contrast, reversible devices are removable and may be fixed or adjustable. A fixed removable device is defined by a non-adjustable ring without an O-ring or topper. In contrast, an adjustable removable device can be interval-based or dynamic-based. Specifically, an interval-based device is defined by a ring with several interchangeable toppers, each with different aperture sizes corresponding to different volumes of gastric outflow. Different toppers can also include different size and/or configurations of the aperture, such as apertures that extend to or partially define an outer perimeter of the gastric outflow device. This can allow medication to pass through more easily, for example. In contrast, a dynamic-based device features a ring with a topper that has an adjustable aperture. Each in different embodiments, the aperture (e.g., size) is adjustable wirelessly, percutaneously, by ultrasonic motor, and/or by including an expandable compartment surrounding the aperture, the expandable compartment increasing and decreasing the gastric outflow device aperture on being filled with filling material and having the filling material extracted, respectively.

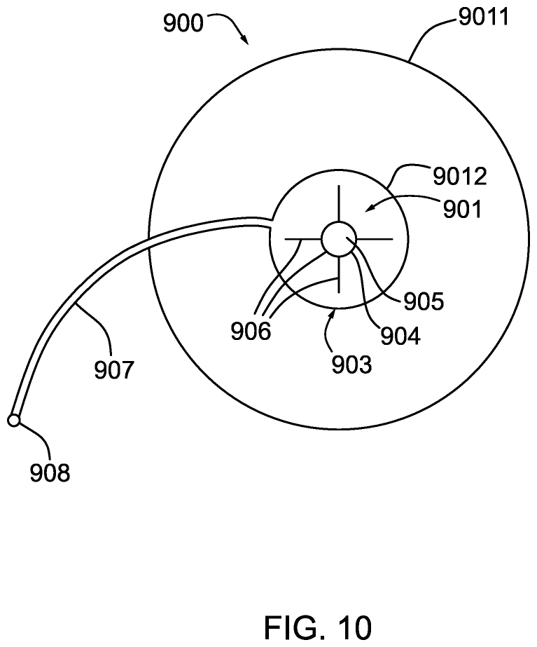

FIG. 10 shows a further embodiment. As shown, topper 900 is defined by a concentric outer perimeter 9011 and inner perimeter 9012. Within the inner perimeter lies an expandable compartment 901 made of suitable elastic material. Expandable compartment 901 can be a bubble. Expandable compartment 901 can be an elastic compartment. Expandable compartment 901 is adjustable (e.g., by filling and/or extracting filling material from within) to define an expandable compartment aperture 905. On assembly of the gastric outflow device, gastric outflow is receivable through the expandable compartment aperture 905. For example, gastric outflow aperture 905 can be aligned with a ring aperture. Expandable compartment 901 can contain a filling material, such as saline, and be filled with saline. The expandable compartment 901 has an outer perimeter 903, inner perimeter 904, and an expandable compartment aperture 905. In the embodiments shown, attached to expandable compartment 901 are springs 906 arranged in a cross formation. In various embodiments, springs 906 may be another suitable support structure. As used herein, references to springs 906 can be substituted with another suitable support structure in different embodiments. In some embodiments, each spring 906 has one end attached to the wall of the expandable compartment 901 forming the outer perimeter 903 and the other end attached to the wall of the expandable compartment 901 forming the inner perimeter 904. A portion of the expandable compartment 901 is connected to a conduit 907 (e.g., tube) that can be positioned to extend to outside the body, such as through the walls of the stomach and abdominal cavity. Conduit 907 may be made of silicone material. The amount of gastric outflow is varied by adjusting the volume of filling material (e.g., saline) in expandable compartment 901. For example, saline solution that is added externally via, for example, a syringe, will flow through the conduit 907 and into the expandable compartment 901, and hence, the volume of the filling material in the expandable compartment 901 increases, narrowing the aperture 905, and thereby decreasing the amount of gastric outflow. Conversely, to increase the amount of gastric outflow, a suction force can be applied to the end of the conduit 907 outside the body to draw filling material from the expandable compartment 901, and hence, the expandable compartment 901 will deflate causing the aperture 905 to widen, and thereby increasing the amount of gastric outflow. Aperture 905 can accordingly be opened and closed. A valve 908 attached to the end of the conduit 907 outside the body can be closed to maintain the pressure in the tube 907 and prevent the backflow of saline. The expansion and contraction of the expandable compartment 901 occurs mostly radially, that is, along the radius of the aperture 905 or device. For example, this can be parallel to the direction of expansion or contraction of the springs 906. Springs 906 can be positioned and oriented to help direct the expansion and contraction of bubble 901 generally to be radial movement. In some embodiments, springs 906 can be replaced with a similarly suitable component or components that can help direct the expansion and contraction of expandable compartment 901 similarly.

In some embodiments, a cross-linking component is attached to a top portion of expandable compartment 901 and to a bottom portion of expandable compartment 901, such as axially through the aperture 905 (e.g., parallel to an axis through expandable compartment 901 in the axial direction). This can reduce or prevent expansion of expandable compartment 901 in the perpendicular or axial direction (as would be depicted in FIG. 10 as being into and out of the page). In some embodiments, there is minimal expansion and contraction of the material-filled expandable compartment perpendicularly, that is, as would be depicted in FIG. 10 into and out of the page), as such movement is restricted by two cross-linkages positioned on the bubble 901. As weight loss is a dynamic process, topper 900 which contains a centrally positioned material-filled expandable compartment can help enable frequent, less invasive, and more cost-effective adjustments of gastric outflow volume according to an individual's weight loss goals. In some embodiments, expandable compartment 901 is another suitable expandable compartment other than a bubble.

In some embodiments, topper 900 includes a silicone tubing 907 that is about 1-2 mm in diameter that extends to outside of the body for aperture adjustment and through which saline or other suitable material is added or removed. Such addition or removal reduces or increases the aperture size 905, respectively, and allows, respectively, less or more gastric components to flow from the stomach to the bowels. This can be used to control caloric intake. An elastic component (e.g., compartment) expands or contracts along the radius of the device as shown. In some embodiments, springs 906 are adjustable using a signal (e.g., electromagnetic signal or positive/negative charges).

In some embodiments, a post-pylorus device wraps around a body conduit (e.g., duodenum) and controls its outflow via control of pressure in the device through a decrease or increase of saline injected into a tube attached to the device. The tube is accessible at body surface for easy access and control.

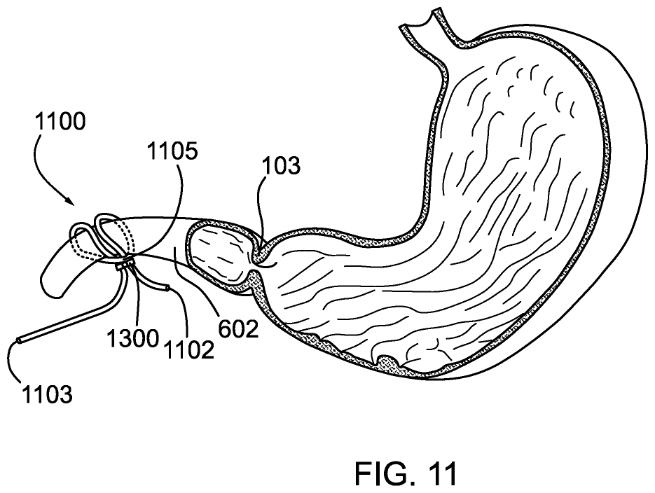

FIG. 11 shows gastric outflow device 1100, according to some embodiments. This can be a post-pylorus gastric outflow device 1100. Gastric outflow device 1100 is defined by a flexible conduit (e.g., tube). Gastric outflow device 1100 can be arranged in a knot with two terminal ends 1102 and 1103 and includes a lock 1300 that secures the knot. For example, the knot is created by folding the tube together and placing the two terminal ends, 1102 and 1103, through the loop 1105. Loop 1105 can be U-shaped. The terminal ends, 1102 and 1103, and the loop 1105 are secured together by lock 1300. Gastric outflow device 1100 can be wrapped around the duodenum 602, which is located post pyloric valve. Terminal end 1102 is sealed and located within the abdominal cavity, while terminal end 1103 remains open and can extend to outside the body. Filling material, such as saline, can be inserted through terminal end 1103 and extracted from gastric outflow device 1100 through terminal end 1103. Terminal end 1103 can be longer than terminal end 1102. The tube may be made of silicone material, while the lock may be made of suitable silicone or plastic material. Gastric outflow device 1100 can be used to vary the amount of gastric outflow, post pyloric valve, to the latter parts of the duodenum. The amount of gastric outflow is varied by adjusting the volume of filling material contained within 1100.

In some embodiments, the portion of gastric outflow device 1100 wrapped around the bodily conduit (e.g., duodenum) is flexible and expandable by injection of filling material (e.g., saline). For example, loop 1105 of the knot is flexible and expandable by injection of filling material. In some embodiments, on injection of saline, loop 1105 expands or balloons to apply pressure to the duodenum.

For example, in some embodiments, a gastric outflow device includes a flexible conduit wrappable around a bodily conduit, the flexible conduit configured to expand (e.g., balloon) to tighten around the bodily conduit following receiving a filling material, the flexible conduit configured to contract (e.g., relax) to loosen around the bodily conduit following extraction of the filling material. A locking mechanism ensures expansion/contraction concentrates the pressure around the conduit hence regulating the flow through the conduit, for example. In some embodiments, a gastric outflow device tightens around the bodily conduit and loosens around the bodily conduit. This can be used to help regulate (e.g., restrict or allow) movement of material through the bodily conduit.

In some embodiments, a fixed diameter wrap can be used in place of the flexible and expandable portion of gastric outflow device 1100. For example, loop 1105 can be a fixed diameter and not expandible. In some embodiments, such a device does not include a tube that receives and allows extraction of filling material. This can create a bottle neck inside the duodenum (or other bodily conduit in some embodiments) increasing resistance and pressure on the duodenal side of the pylorus, facilitating its closure (or more modest opening) and facilitating regulation of outflow from the stomach.

Lock 1300 can help secure gastric outflow device 1100 such that gastric outflow device 1100 is wrapped securely around the duodenum and the gastric outflow device 1100 (e.g., at the loop 1105) is kept at a minimal size (e.g., in a tight fit around the portion of the body that gastric outflow device 1100 is secured around) in order to maintain pressure within gastric outflow device 1100 as filling material is introduced. For example, to decrease the amount of gastric outflow, saline solution can be added via external terminal end 1103, leading to increased volume of saline solution within gastric outflow device 1100, and cause gastric outflow device 1100 to expand and loop 1105 to tighten against the duodenum 602. This tightening of the loop 1105 decreases the diameter of the duodenum portion wrapped by gastric outflow device 1100, and, as a result, reduce gastric outflow and lead to increased pressure in the stomach, which may contribute to the feeling of satiety. The loop 1105 is expandable to accommodate duodenums of different widths; and is extendable along the length of the duodenum to vary the length of the duodenum to be restricted. In an embodiment, gastric outflow device 1100 may be used in conjunction with another gastric outflow device embodiment, such as gastric outflow device 100, to control gastric outflow post pyloric valve and pre pyloric valve, respectively. In some other embodiments, gastric outflow device 1100 may be wrapped around a different portion of the digestive tract, where suitable, to control gastric outflow. FIG. 11 shows lock 1300 prior to being secured under loop 1105.

In some embodiments, gastric outflow device 1100 is arranged in a different configuration around a portion of the body, such as without a loop 1105. For example, a different shaped loop can be used. Lock 1300 is positioned to secure gastric outflow device 1100 around the portion of the body so as to create a suitable fit and allow the portion of the body to be constricted and expanded accordingly on insertion and/or removal of filling material from gastric outflow device 1100. In some embodiments, gastric outflow device 1100 can help control pressure on the duodenum using saline hence controlling outflow from the stomach and satiety. Gastric outflow device 1100 can be used as a pre-pyloric valve or post-pyloric valve device.

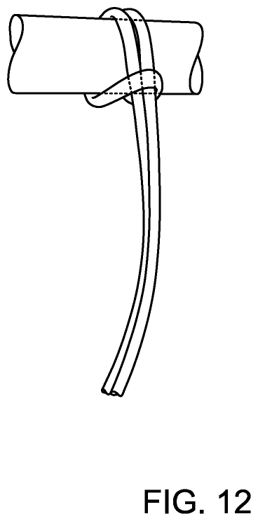

FIG. 12 shows a schematic view of an example gastric outflow device 1100, according to some embodiments. This can be a post-pylorus gastric outflow device. Tubing of the gastric outflow device 1100 is only partially shown.

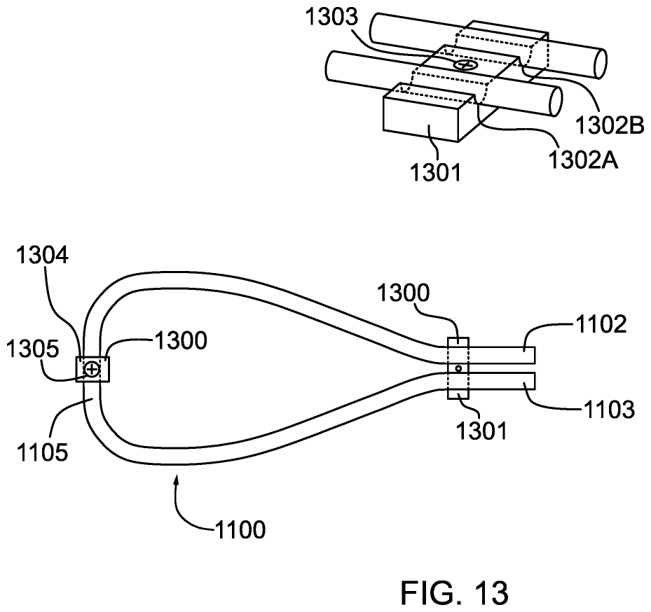

FIG. 13 shows lock 1300 used to hold portions of the gastric outflow device 1100 that extend to terminal ends 1102 and 1103 and a portion of the gastric outflow device 1100 (e.g., loop 1105) together, according to some embodiments. According to some embodiments, as shown in FIG. 13, the lock 1300 is defined by a rectangular shaped block 1301 with two indentations 1302A and 1302B that lie parallel to the width of the block and a screw 1303 between the two indentations; and another screw 1305 in square shaped block 1304. The portions of the gastric outflow device 1100 leading to terminal ends 1102 and 1103 each lie within an indentation 1302. The square shaped block 1304 containing screw 1305 can be attached to the complementary portion of lock 1300 (e.g., block 1301) that is configured at another portion of gastric outflow device 1100 (e.g., at loop). The two blocks 1301 and 1304, and the two screws 1303 and 1305, are complementary, such that when they contact each other, the terminal ends 1102 and 1003 attach securely to the loop 1105, forming a knot. In some embodiments, other attachment mechanisms other than lock 1300 can be used.

According to various embodiments, the gastric outflow device is attachable between the stomach and the intestine, such as on the stomach side or on the duodenum side. The gastric outflow device can be attached to the pyloric sphincter, for example.

Various embodiments of the invention have been described in detail. Since changes in and or additions to the above-described best mode may be made without departing from the nature, spirit or scope of the invention, the invention is not to be limited to those details but only by the appended claims.

What is claimed is:

1. A gastric outflow device, comprising:
a ring having a ring aperture; and
a topper having a topper aperture, the topper attachable to the ring, the topper aperture alignable with the ring aperture to form the gastric outflow device aperture.

2. The gastric outflow device of claim 1, the topper comprising screws spaced near an outer perimeter of the topper, the ring comprising holes alignable for receiving the screws.

3. The gastric outflow device of claim 2, the topper further comprising protrusions spaced along a surface of the topper, the ring further comprising grooves alignable for receiving the protrusions.

4. The gastric outflow device of claim 3, the screws evenly spaced along the outer perimeter of the topper and the protrusions evenly spaced between the screws.

5. The gastric outflow device of claim 1, the gastric outflow device aperture extending to an outer perimeter of the gastric outflow device.

6. The gastric outflow device of claim 1, the gastric outflow device aperture defining a portion of an outer perimeter of the gastric outflow device.

7. The gastric outflow device of claim 1, the topper aperture adjustable by wireless signal.

8. The gastric outflow device of claim 1, the topper aperture adjustable by percutaneous control.

9. The gastric outflow device of claim 1, the topper aperture adjustable by an ultrasonic motor.

10. The gastric outflow device of claim 1, the ring further comprising staples attachable to a pyloric sphincter and the topper further comprising screws securable to the ring.

11. The gastric outflow device of claim 1, further comprising an expandable compartment expandable by being filled with filling material and contractable by extraction of the filling material, the expandable compartment positioned in the gastric outflow device aperture and comprising an expandable compartment aperture.

12. The gastric outflow device of claim 11, further comprising a conduit in fluid communication with the expandable compartment, the conduit comprising a valve through which the filling material is insertable and extractable.

13. The gastric outflow device of claim 11, further comprising at least one support attachable to an inner perimeter of the expandable compartment and to a separate portion of the expandable compartment, the inner perimeter defining the expandable compartment aperture, the at least one support directing expansion or contraction of the expandable compartment radially.

14. The gastric outflow device of claim 13, further comprising a cross-linking structure attachable to opposing surfaces of the expandable compartment, the cross-linking structure restricting movement of the expandable compartment axially.

15. A method for restricting gastric outflow, comprising:
directing gastric outflow through an aperture sized and dimensioned according to a pre-determined level of gastric outflow desired.

16. The method of claim 15, further comprising adjusting the size of the aperture based on an adjusted pre-determined level of gastric outflow desired.

17. The method of claim 16, the adjusting further comprising an elastic adjustment by filling or extracting filling material surrounding the aperture.

18. The method of claim 15, further comprising replacing the aperture with a replacement aperture sized and dimensioned according to an adjusted pre-determined level of gastric outflow desired.

19. The method of claim 15, further comprising adjusting the size of the aperture using percutaneous control.

20. A gastric outflow device, comprising:
a flexible conduit wrappable around a bodily conduit, the flexible conduit configured to expand to tighten around the bodily conduit following receiving a filling material, the flexible conduit configured to contract to loosen around the bodily conduit following extraction of the filling material.
